(19) United States
(12) Patent Application Publication
(10) Pub. No.: US 2008/0275504 A1
(43) Pub. Date: Nov. 6, 2008

(54) Constructs for dynamic spinal stabilization

(76) Inventors: Henry K. Bonin, Memphis, TN (US); Aurelian Bruneau, Memphis, TN (US); Thomas Carls, Memphis, TN (US); Eric C. Lange, Collierville, TN (US); John D. Pond, Germantown, TN (US); Kent M. Anderson, Memphis, TN (US)

Correspondence Address: KRIEG DEVAULT LLP, ONE INDIANA SQUARE, SUITE 2800, INDIANAPOLIS, IN, 46204-2709, US

(21) Appl. No.: 11/799,678

(22) Filed: May 2, 2007

(51) Int. Cl. A61B 17/70; A61B 17/04

(52) U.S. Cl. 606/246; 606/301; 606/305

(57) ABSTRACT

Devices and methods for spinal stabilization include first and second anchors engageable to respective ones of first and second vertebrae and a connector assembly engageable with the anchors to provide a desired stabilization effect. The connector assembly includes a pair of bumper assemblies extending between the bone anchors and a connecting element extending through the bumper assemblies and connecting the first and second bone anchors to one another.

### BACKGROUND

Elongated connecting elements, such as rods, plates, tethers, wires, cables, and other devices have been implanted along the spinal column and connected between two or more anchors engaged between one or more spinal motion segments. Such connecting elements can provide a rigid construct that resists movement of the spinal motion segment in response to spinal loading or movement of the spinal motion segment by the patient. Still other connecting elements are flexible to permit at least limited spinal motion while providing resistance to loading and motion of the spinal motion segment. Such flexible connecting elements can be considered to provide dynamic spinal stabilization since at least limited movement of the spinal motion segment is preserved after implantation of the connecting element.

While prior connecting elements provide various spinal stabilization options, there remains a need for stabilization constructs that can provide dynamic resistance to forces and permit motion of the spinal column segment while maintaining stabilization of the spinal column segment and the structural integrity of the construct.

### SUMMARY

The present invention generally relates to constructs and methods for dynamically stabilizing a spinal column motion segment including at least two vertebrae by engaging the construct between the at least two vertebrae. The construct can be engaged to at least two anchors engaged to respective ones of the at least two vertebrae while permitting motion of the vertebrae relative to one another. The construct includes at least two bumper elements extending between the anchors to resist movement of the anchors toward one another and a connecting element extending between the anchors to axially link the anchors to one another.

According to one aspect, a spinal stabilization construct comprises first and second bone anchors and a connector assembly extending along a longitudinal axis between proximal receiver portions of the first and second bone anchors. The connector assembly includes first and second mounting elements positioned in the passage of a respective one the first and second bone anchors. Each of the mounting elements includes a body extending along the longitudinal axis between an inner end located between the first and second bone anchors and an opposite outer end located on an opposite side of the respective first and second bone anchor. The connector assembly also includes first and second intermediate bumper elements extending from a respective one of the inner ends of the mounting elements. The intermediate bumper elements have end portions in abutting engagement with one another between the first and second mounting elements. The connector assembly also includes first and second outer bumper elements adjacent a respective one of the outer ends of the first and second mounting elements. The connector assembly also includes an elongated connecting element extending through the mounting elements and the intermediate bumper elements. The connecting element is secured to the outer bumper elements.

According to a further aspect, a spinal stabilization construct comprises first and second bone anchors and a connector assembly extending along a longitudinal axis between proximal receiver portions of the first and second bone anchors. The connector assembly includes first and second mounting elements positioned in the passage of a respective one of the first and second bone anchors. Each of the mounting elements includes a body extending along the longitudinal axis between an inner end located between the first and second bone anchors and an opposite outer end. The connector assembly also includes first and second intermediate bumper elements extending from a respective one of the first and second mounting elements. Each of the first and second intermediate bumper elements includes an inner end in abutting engagement with the inner end of the other of the first and second intermediate bumper elements. The connector assembly also includes a connecting element extending through the mounting elements and the intermediate bumper elements. The connecting element is elastic and tensioned to maintain a compression force between the first and second bone anchors and the first and second intermediate bumper elements.

According to another aspect, a method for assembling a spinal stabilization construct, comprises: providing a first bone anchor spaced from a second bone anchor; positioning a first connector portion on the first bone anchor and a second connector portion on the second bone anchor, wherein each of the first and second connector portions includes respective ones of first and second intermediate bumper elements extending to an inner end situated between the first and second bone anchors; providing a connecting element extending through the first and second connector portions; and tensioning the connecting element and securing it to compress the first and second bone anchors with the inner ends of the first and second intermediate bumper elements in abutting and pivotal contact with one another.

These and other aspects will be discussed further below.

### BRIEF DESCRIPTION OF THE DRAWINGS

FIG. 1 is a side section view a spinal column segment with a dynamic stabilization construct secured thereto.

FIG. 2 is a longitudinal section view of the stabilization construct of FIG. 1.

FIG. 3 is a section view along line 3-3 of FIG. 1.

FIG. 4 is a section view along line 4-4 of FIG. 1.

FIG. 5 is a perspective view of an outer bumper element of the stabilization construct of FIG. 1.

FIG. 6 is a perspective view of an intermediate bumper element of the stabilization construct of FIG. 1.

FIG. 7A is a perspective view of a securing member of the stabilization construct of FIG. 1.

FIG. 7B is a perspective view of another embodiment securing member of the stabilization construct of FIG. 1.

FIGS. 8A-8C are elevation views of various axial arrangements of connector portions of the connector assembly.

FIG. 9 is a side section view a spinal column segment with another embodiment dynamic stabilization construct secured thereto.

FIG. 10 is a longitudinal section view of another embodiment stabilization construct and a portion of a bone anchor.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

Constructs and methods for providing dynamic stabilization of one or more spinal motion segments are provided. The constructs and methods include a connector assembly between two or more bone anchors that are engaged to respective ones of at least two or more vertebral bodies of a spinal motion segment. The connector assembly includes a pair of connector portions engaged to respective ones of the bone anchors and each with an intermediate bumper element located between the bone anchors to dynamically resist movement of the anchors toward one another. The connector assembly also includes a connecting element extending through the intermediate bumper elements and the anchors. The connecting element is coupled to outer bumper elements located on an outer side of the respective anchor, and the connecting element axially couples the anchors to one another to dynamically resist movement of the anchors away from one another.

The connector portions can each be formed or assembled to provide a single construct and engaged to the respective anchor to form the connector assembly. The connector portions can each include multiple components assembled to provide various lengths that are employed to provide the desired length between the anchors. The components of the respective connector portions can be formed together by fusion, molding, fasteners, adhesives or other securing technique or system.

The bone anchors discussed herein can be multi-axial or uni-axial in form, and can include an anchor portion engageable to a vertebral body or other bony structure and a proximal receiver portion for receiving or engaging a respective end of the connector assembly. The multi-axial anchors allow the anchor portion to be positioned at various angles relative to the receiver portion of the anchor. The uni-axial anchors provide a fixed positioning of the receiver portion to the anchor portion. The anchor portion of the bone anchors includes a distal lower portion that is engageable to a vertebral body with the proximal receiver portion positioned adjacent to the vertebral body. In one embodiment, the anchor portion is in the form of a bone screw with a threaded shaft and the proximal receiver portion includes a passage for receiving the connector assembly. The receiver portion is fixed relative to the shaft in one embodiment, and pivotal relative to the shaft in another embodiment. In other embodiments, the distal anchor portion is in the form of a hook, staple, cable, tether, suture anchor, interbody fusion implant, artificial disc implant, bolt, or other structure engageable to bony structure. The passage of the receiver portion can define a U-shape, O-shape, or other shape that defines a passage that receives or engages the respective end of the connector assembly therein, thereon, therethrough, or thereover, for example. The connector assembly can extend from one or both of the bone anchors for securement to one or more additional vertebral bodies in multi-level stabilization constructs.

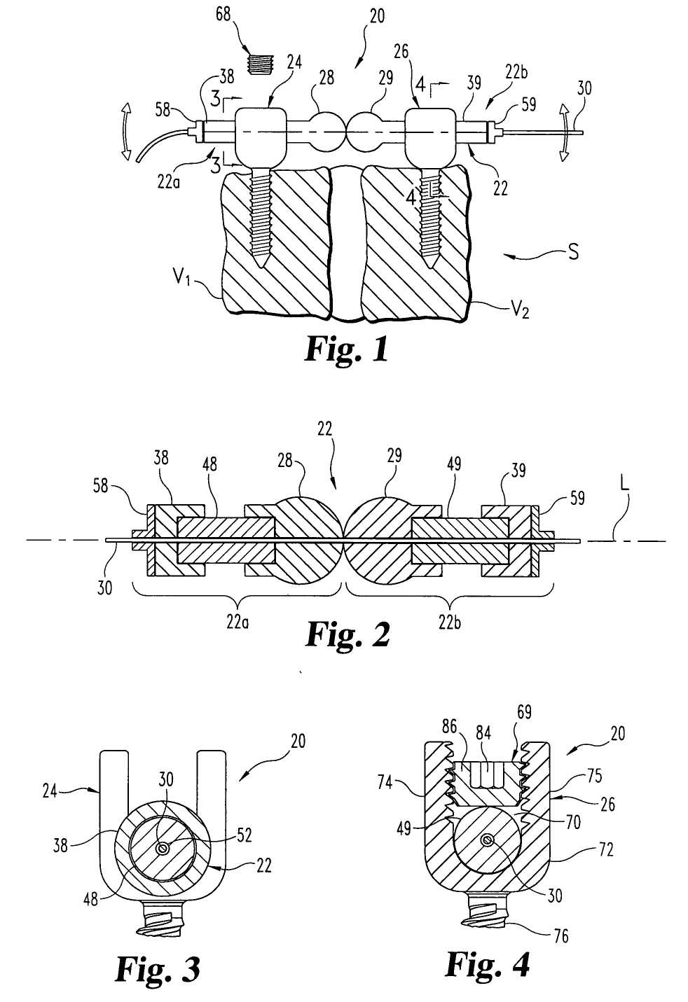

FIG. 1 illustrates a dynamic stabilization construct 20 engaged along a spinal column of a patient with the spinal column shown diagrammatically and in longitudinal section. In one embodiment, stabilization construct 20 is affixed to pedicles of vertebrae V1 and V2 of the spinal column segment S from a posterior approach. Also contemplated are applications in posterior-lateral, lateral, antero-lateral and anterior approaches, and applications where the stabilization construct 20 is engaged to other portions of the vertebrae V1 and V2, such as the anterior body portion or any of the posterior elements of the vertebrae. The spinal column segment S can comprise two vertebrae V1, V2 as shown for a single level stabilization procedure or three or more vertebrae in multi-level stabilization procedures. The vertebrae V1, V2 can be any one or combination of the sacral, lumbar, thoracic, and/or cervical vertebrae of the spinal column.

Stabilization construct 20 includes a connector assembly 22 extending along a longitudinal axis L between first bone anchor 24 and second bone anchor 26. Connector assembly 22 includes first and second connector portions 22a, 22b having first and second intermediate bumper elements 28, 29, respectively, positioned between bone anchors 24, 26 and in contact with one another to dynamically resist movement of bone anchors 24, 26 toward one another. Connector portions 22a, 22b of connector assembly 22 also include respective outer bumper elements 38, 39 located adjacent to respective ones of the bone anchors 24, 26 on the side thereof opposite respective ones of the intermediate bumper elements 28, 29. Outer bumper elements 38, 39 dynamically resist movement of the bone anchors 24, 26 away from one another.

Connector portions 22a, 22b of connector assembly 22 also include mounting elements 48, 49 (FIG. 2) extending through respective ones of the bone anchors 24, 26. Intermediate bumper elements 28, 29 are located on the respective inner ends of mounting elements 48, 49 and outer bumper elements 38, 39 are located on the respective outer ends of mounting elements 48, 49. Connector assembly 22 also includes a connecting element 30 extending along axis L through the mounting elements 48, 49 and bumper elements 28, 29. Connecting element 30 is secured to outer bumper elements 38, 39 with respective ones of the first and second securing members 58, 59 located adjacent to and in abutting engagement with the outer ends of the adjacent outer bumper element 38, 39. Mounting elements 48, 49 are engaged in the respective bone anchor 24, 26 with respective ones of the first and second engaging members 68, 69 to secure mounting elements 48, 49 in position relative to the respective bone anchor 24, 26.

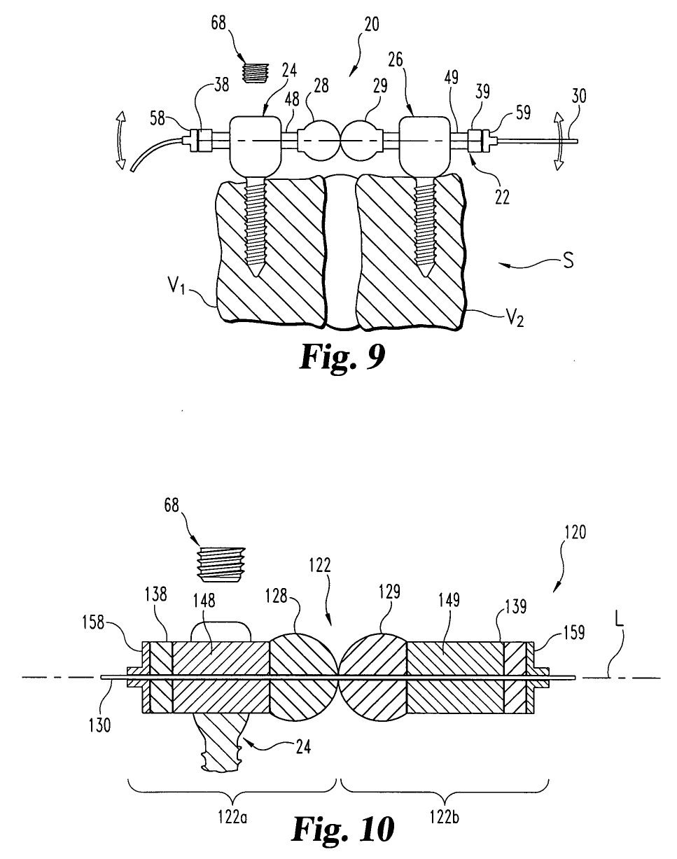

Connector assembly 22 includes connector portions 22a, 22b that provide a pair of bumper assemblies engaged to respective ones of first and second bone anchors 24, 26 and linked to one another with connecting element 30. Connector portion 22a provides a first bumper assembly that includes mounting element 48, intermediate bumper element 28 on an inner end of mounting element 48, and an outer bumper element 38 on an outer end of mounting element 48. Mounting element 48 includes a mounting length between bumper elements 28, 38 to provide a rigid interface with bone anchor 24. In one embodiment, bumper elements 28, 38 abuttingly engage opposite sides of bone anchor 24 at its receiver portion such as shown in FIG. 1. In another embodiment, mounting element 48 is provided with sufficient length so that one or both of bumper elements 28, 38 is axially spaced from bone anchor 24, such as shown in FIG. 9.

Connector portion 22b provides a second bumper assembly that includes mounting element 49, intermediate bumper element 29 on an inner end of mounting element 49, and an outer bumper element 39 on an outer end of mounting element 49. Mounting element 49 includes a mounting length between bumper elements 29, 39 to provide a rigid interface with bone anchor 26. In one embodiment, bumper elements 29, 39 abuttingly engage opposite sides of bone anchor 26 at its receiver portion. In another embodiment, mounting element 49 is provided with sufficient length so that one or both of bumper elements 29, 39 is axially spaced from bone anchor 26, such as shown in FIG. 9. The location along mounting elements 48, 49 to which the respective anchor 24, 26 is engaged can be varied to accommodate the spacing between anchors 24, 26.

Connecting element 30 is engaged to outer bumper elements 38, 39 to axially link bone anchors 24, 26 and resist movement of bone anchors 24, 26 away from one other. Connecting element 30 is flexible and tensionable and outer bumper elements 38, 39 are compressible to allow at least limited movement of bone anchors 24, 26 away from one another in response to spinal motion. Intermediate bumper elements 28, 29 are positioned in abutting engagement and compressible to allow at least limited motion of bone anchor 24, 26 toward one another in response to motion of the spinal column segment. The bumper elements and connecting element are resilient to normally return toward a pre-stressed configuration as anchors 24, 26 are returned to a neutral position.

Connector assembly 22 includes an overall length along longitudinal axis L sized to extend between bone anchors 24, 26 when engaged to at least two vertebral bodies V1, V2. In stabilization construct 20, bone anchors 24, 26 are affixed to various locations of the spinal column segment S, such as the pedicles, and interconnected with one or more connector assemblies 22. Other procedures contemplate connector assemblies 22 may be employed at other locations about the spinal column, including anterior, antero-lateral, and lateral locations. Stabilization construct 20 may also be employed in procedures where such locations are combined; e.g. to provide posterior and anterior stabilization. Stabilization construct 20 may be used for, but is not limited to, treatment of degenerative spondylolisthesis, herniation, degeneration, arthritis, fracture, dislocation, scoliosis, kyphosis, spinal tumor, and/or a failed previous fusion.

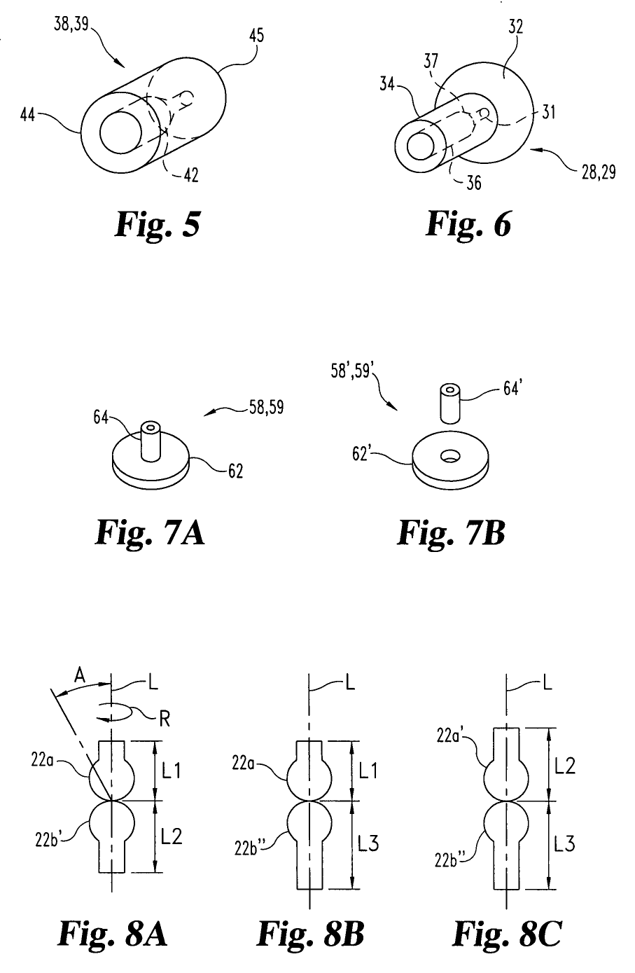

FIGS. 3-4 show section views through FIG. 1 at various locations along the connector assembly 22. In FIG. 5 outer bumper elements 38, 39 are shown with axial passage 42 extending therethrough and opening at opposite ends 44, 45 of outer bumper elements 38, 39. At least a portion of passage 42 is sized and shaped to receive the respective mounting element 48, 49 therein, as shown in FIGS. 2 and 3. The remaining portion of passage 42 is sized and shaped to receive connecting element 30 therethrough. Outer bumper elements 38, 39 include a cylindrical shape with an outer circular cross-sectional profile. Other embodiments contemplate other cross-sectional shapes, including non-circular shapes, for outer bumper elements 38, 39.

Connecting element 30 also extends through an axial passage 52 extending through mounting element 49. Outer bumper element 38 includes a cylindrical shape extending around the outer end of mounting element 48. Outer bumper element 38 is configured to axially slide about mounting element 48 in one embodiment. In another embodiment, outer bumping element 38 is integrally formed with mounting element 48, or can be axially fixed with mounting element with an adhesive, fasteners, fusion, or over-molding fabrication technique. Outer bumper element 39 can be similarly arranged relative to mounting element 49.

In FIG. 4 mounting element 49 is situated in passage 70 of bone anchor 26. Bone anchor 26 includes proximal receiver portion 72 that has a pair of arms 74, 75 extending along opposite sides of passage 70. Anchor portion 76 extends distally from receiver portion 72 for engagement with the bony structure. In the illustrated embodiment, anchor portion 76 is integrally formed with and fixed with receiver portion 72. Other embodiments contemplate that receiver portion 72 is pivotal and/or rotatable relative to anchor portion 76 to provide a multi-axial type screw arrangement that allows the passage 70 be positioned in a desired alignment even when the anchor portion 76 is engaged to the bony structure. Arms 74, 75 are internally threaded to threadingly receive engaging member 68, 69. In other embodiments, arms 74, 75 include external threads, a slot arrangement, or any suitable configuration to engage any suitable engaging member.

Engaging members 68, 69 are engaged to internal threads along arms 74, 75 and positioned in contact with mounting element 49 in passage 70 to secure mounting element 49 in receiver portion 72. In one embodiment, engaging member 69 fixes mounting element 49 in passage 70. Connecting element 30 extends through mounting element 49 and is axially movable relative to mounting element 49 and bone anchor 26 even when mounting element 49 is fixed in receiver portion 72. Mounting element 48 can be similarly engaged in the passage of the receiver portion of bone anchor 24 with engaging member 68. In other embodiments, one or both of the mounting elements are axially movable and/or pivotal in the passage of the respective bone anchor.

Engaging members 68, 69 include a proximal tool engaging recess 84 and a shaft portion 86 in the illustrated embodiment. Shaft portion 86 is shown in the form of a set screw to threadingly engage arms 74, 75. Engaging members 68, 69 can be provided with a removable proximally extending tool engaging portion (not shown) that is severable from shaft 86 upon application of a threshold torque. Other forms for engaging members 68, 69 are also contemplated, including nuts, caps, plugs, and sliding locking elements, for example.

FIG. 6 shows intermediate bumper elements 28, 29 in an isolated perspective view. Bumper elements 28, 29 include an end portion 32 forming a spherical shape, and an extension portion 34 extending axially from end portion 32. Extension portion has a cylindrical shape defining a circular cross-section about longitudinal axis L. Other shapes for extension portion 34 are also contemplated, including oval, square, or irregular shapes. Other shapes for end portions 32 are also contemplated, including non-spherical shapes that are enlarged relative to cylindrical portion 34, or shapes that are similar in size or smaller in size than cylindrical portion 34.

Intermediate bumper elements 28, 29 include axial passage 36 opening at an end of extension portion 34 and extending in extension portion 34 to an end 37 in or adjacent to end portion 32. Passage 36 is sized to receive the respect mounting element 48, 49 therein. An end passage 31 extends through end portion 32 and is in communication with axial passage 36. End passage 31 is sized to receive connecting element 30 therethrough and allow connecting element 30 to extend through intermediate bumper elements 28, 29 and continuously along the length of the connector assembly 22. As discussed above, intermediate bumper elements 28, 29 can be removably positioned around respective ones of the mounting elements 48, 49 or fixed thereto with an adhesive, fastener, fusion, or over-molding, or other fabrication technique.

FIG. 7A shows a perspective view of securing members 58, 59. Securing member 58, 59 include an enlarged platform portion 62 and a securing portion 64 extending from platform portion 62. Platform portion 62 resides in abutting engagement with a respective adjacent end of outer bumper elements 38, 39 to distribute axial compression loading over all or nearly all of the surface area thereof. Connecting element 30 extends through platform portion 62 and securing portion 64. Securing portion 64 can be crimped, deformed, or otherwise fastened with connecting element 30 to hold connector assembly 22 in firm engagement with the bone anchors 24,26 and normally maintain connecting element 30 in tension in the absence of compression loading of the intermediate bumper elements 28, 29.

In FIG. 7A, securing portion 64 is integrally formed with platform portion 62, and includes a cylindrical shape extending from the disc shaped platform portion that is crimped around connecting element 30. In FIG. 7B another embodiment of securing members 58′, 59′ is shown with securing portion 64′ separated from platform portion 62′. Securing portion 64′ is assembled in abutting engagement against platform portion 62′ with connecting element 30 extending through platform portion 62′ and securing portion 64′.

Referring back to FIGS. 1 and 2, when assembled, connector assembly 22 is positioned between bone anchors 24,26 so that mounting elements 48, 49 are located in the receiver portions of the respective bone anchors 24, 26. Intermediate bumper elements 28, 29 are axially located between bone anchors 24, 26. In one embodiment, the extension portions 34 are in contact with the respective receiver portion of the bone anchors and with the spherical end portions 32 in abutting engagement in end-to-end relation. In another embodiment, one or both of extension portions 34 are axially spaced from the respective adjacent bone anchor 24, 26. In one embodiment, outer bumper elements 38, 39 are located axially along the respective mounting element 48, 49 in contact with receiver portion of the bone anchors 24, 26 so that bone anchor 24 is positioned between bumper elements 28, 38 and bone anchor 26 is positioned between bumper elements 29, 39. In other embodiments, one or both of outer bumper elements 38, 39 are axially spaced from the receiver portion of the adjacent bone anchor 24, 26.

One of the securing elements 58, 59 is secured to connecting element 30 and in abutting engagement with the respective adjacent end of the adjacent outer bumper element 38, 39. Connecting element 30 is then tensioned so that the intermediate bumper elements 28, 29 are in abutting engagement with one another, and the other securing member 58, 59 is secured to connecting element 30 to maintain the assembled positioning of the components of connector assembly 22. Engaging members 68, 69 are engaged to the respective bone anchor 24, 26 to engage mounting elements 48, 49 in the coupling assembly either prior to or after tensioning of connecting element 30.

The spacing between bone anchors 24, 26 can be readily adjusted by selecting connector portions of the desired individual length to provide a desired overall length for the connector assembly between bone anchors 24, 26. The change in length can be selected to provide a desired angle between the endplates of the vertebrae to which bone anchors 24, 26 are engaged to restore or maintain, for example, lordosis of the spinal motion segment. FIGS. 8A-8C show various intermediate bumper element arrangements of varying length. In FIGS. 1 and 2, connector portions 22a, 22b have the same length L1 along longitudinal axis L. In FIG. 8A, an alternate connector portion 22b is shown with a length L2 along longitudinal axis L that is greater than length L1 of connector portion 22a. In FIG. 8B, another connector portion 22b″ is provided with a length L3 along longitudinal axis that is greater than length L2.

The connector portions are selected and arranged to provide the desired overall length between anchors 24, 26. The connector assembly including connector portions 22a, 22b′ in FIG. 8A has an overall length L1, L2 that is greater than a connector assembly that includes connector portions 22a, 22b each of length L1. The connector assembly with connector portions 22a, 22b″ in FIG. 8B has an overall length L1, L3 that is greater than a connector assembly that includes connector portions 22a, 22b′. The connector assembly with connector portions 22a′, 22b″ in FIG. 8C has an overall length L2, L3 that is greater than a connector assembly that includes bumper elements 22a, 22b″. In still other connector assemblies, the overall length is varied by providing connector portions 22a′, 22b′ each with length L2, or by providing an assembly with connector portions that each have length L3 as indicated by connector portion 22b″.

FIG. 10 shown another embodiment stabilization construct 120 that is similar to construct 20 discussed above. Stabilization construct 120 includes a connector assembly 122 extending along a longitudinal axis L between first bone anchor 24 and a second bone anchor (not shown) such as bone anchor 26 discussed above. Connector assembly 122 includes first and second connector portions 122a, 122b having first and second intermediate bumper elements 128, 129, respectively, positioned between bone anchors 24, 26 and in contact with one another to dynamically resist movement of bone anchors 24, 26 toward one another. Connector portions 122a, 122b of connector assembly 122 also include respective outer bumper elements 138, 139 located adjacent to respective ones of the bone anchors 24, 26 on the side thereof opposite respective ones of the intermediate bumper elements 128, 129. Outer bumper elements 138, 139 dynamically resist movement of the bone anchors 24, 26 away from one another.

Connector portions 122a, 122b of connector assembly 122 also include mounting elements 148, 149 extending through respective ones of the bone anchors 24, 26. Intermediate bumper-elements 128, 129 are located in abutting, end-to-end engagement with the respective inner ends of mounting elements 148, 149 and outer bumper elements 138, 139 are located in abutting, end-to-end engagement with the respective outer ends of mounting elements 148, 149. Connector assembly 122 also includes a connecting element 130 extending along axis L through the mounting elements 148, 149 and bumper elements 128, 129. Connecting element 130 is secured to outer bumper elements 138, 139 in abutting, end-to-end engagement with respective ones of the first and second securing members 158, 159 located adjacent to the outer ends of the adjacent outer bumper element 138, 139. Mounting elements 148, 149 are engaged in the respective bone anchor 24, 26 with respective ones of the first and second engaging members 68, 69 to secure mounting elements 148, 149 in position relative to the respective bone anchor 24, 26.

Connector assembly 122 includes connector portions 122a, 122b that provide a pair of bumper assemblies engaged to respective ones of first and second bone anchors 24, 26 and linked to one another with connecting element 130. Connector portion 122a provides a first bumper assembly that includes mounting element 148, intermediate bumper element 128 at an inner end of mounting element 148, and an outer bumper element 138 at an outer end of mounting element 148. Mounting element 148 includes a mounting length between bumper elements 128, 138 to provide a rigid interface with bone anchor 24. Connector portion 122b provides a second bumper assembly that includes mounting element 149, intermediate bumper element 129 at an inner end of mounting element 149, and an outer bumper element 139 at an outer end of mounting element 149. Mounting element 149 includes a mounting length between bumper elements 129, 139 to provide a rigid interface with bone anchor 26.

Connecting element 130 is engaged to outer bumper elements 138, 139 to axially link bone anchors 24, 26 and resist movement of bone anchors 24, 26 away from one other. Connecting element 130 is flexible and tensionable and outer bumper elements 138, 139 are compressible to allow at least limited movement of bone anchors 24, 26 away from one another in response to spinal motion. Intermediate bumper elements 128, 129 are positioned in abutting engagement and compressible to allow at least limited motion of bone anchor 24, 26 toward one another in response to motion of the spinal column segment. The bumper elements and connecting element are resilient to normally return toward a pre-stressed configuration as anchors 24, 26 are returned to a neutral position. In the illustrated embodiment of FIG. 10, the bumper elements are arranged in axial and abutting end-to-end engagement with respective mounting element. Outer bumper elements 138, 139 abut the respective mounting element 148, 149 to resist movement of the bone anchors 24, 26 away from one another. Intermediate bumper elements 128, 129 abut another to resist movement of bone anchors 24, 26 toward one another. Outer bumper elements 138, 139 and intermediate bumper elements 128, 129 do not abuttingly engage the respective adjacent anchor 24, 26 or extend axially along the respective mounting element 148, 149.

In any of the connector assembly embodiments, the intermediate bumper elements can be removable from mounting elements so that the desired length connector portion can be assembled by the surgeon. In other embodiments, the varying length connector portions are provided with intermediate and outer bumper elements pre-mounted to a corresponding mounting element. The varying lengths of the connector portions can be provided by varying the length of the extension portion 34 extending from the corresponding end portion 32, by increasing the length of the end portions of the intermediate bumper element 48, 49, 148, 149 along longitudinal axis L, by increasing the length of each of the portions 32, 34 along longitudinal axis L, and/or by increasing the length of mounting elements 48, 49 or 148, 149.

When assembled between bone anchors 24, 26, the intermediate bumper elements contact each other in end-to-end relation with the spherical end portions facilitating movement of one of the intermediate bumper elements relative to other in response to movement of the corresponding bone anchors 24, 26 resulting from motion of the vertebral level along which the construct is attached. For example, as shown in FIG. 8A connector portion 22a with bumper element 28 can pivot relative to longitudinal axis L and connector portion 22b′ as indicated by angle A. Rotation of connector portion 22a about longitudinal axis L, in either a pivoted or non-pivoted position, is also possible as indicated by arrow R. In addition to or in alternative to the motion of connector portion 22a, the other connector portion 22b can also pivot relative to longitudinal axis L and rotate about longitudinal axis L in the pivoted or non-pivoted position.

The intermediate and outer bumper elements can be made from a polymer material, such as PEEK, or other suitable material. The bumper elements could be made from silicone material, polyurethane, elastomers, or other material providing the desired load resistance properties and stiffness. The mounting elements are made from any suitable material, including metals, metal alloys, and polymers. The mounting elements are rigid and stiff in one embodiment, while the bumper elements are flexible and compressible. In one embodiment, the outer bumper elements have the same stiffness as the intermediate bumper elements. In another embodiment, the stiffness of the outer bumper elements is less than the stiffness of intermediate bumper elements so that motion of anchors away from one another is greater than motion of anchors toward one another under the same loading conditions. In yet another embodiment, the stiffness of the outer bumper elements is greater than the stiffness of the intermediate bumper elements so that motion of anchors 24, 26 toward one another is greater than motion of anchors 24, 26 away from one another under the same loading conditions.

While the invention has been illustrated and described in detail in the drawings and foregoing description, the same is to be considered as illustrative and not restrictive in character, it being understood that all changes and modifications that come within the spirit of the invention are desired to be protected.

What is claimed is:

1. A spinal stabilization construct, comprising:
first and second bone anchors, said first and second bone anchors each including a proximal receiver portion with a passage and a distal portion engageable to respective ones of first and second vertebral bodies;
a connector assembly extending along a longitudinal axis between said proximal receiver portions of said first and second bone anchors, said connector assembly including:
first and second mounting elements positioned in said passage of a respective one of said first and second bone anchors, each of said mounting elements including a body extending along said longitudinal axis between an inner end located between said first and second bone anchors and an opposite outer end located on an opposite side of said respective one of said first and second bone anchors;
first and second intermediate bumper elements extending from a respective one of said inner ends of said mounting elements, said intermediate bumper elements having end portions in abutting engagement with one another between said first and second mounting elements;
first and second outer bumper elements extending from a respective one of said outer ends of said mounting elements; and
an elongated connecting element extending through said mounting elements and said intermediate bumper elements, wherein said connecting element is secured to said outer bumper elements.

2. The construct of claim 1, wherein said distal portion is a threaded shaft and said receiver portion is integrally formed with said threaded shaft.

3. The construct of claim 1, wherein said distal portion is a threaded shaft and said receiver portion pivotal relative to said shaft.

4. The construct of claim 1, wherein said connecting element is a flexible, elastic and non-rigid member.

5. The construct of claim 4, further comprising first and second securing members including a disc portion in abutting engagement with outer ends of each of said respective adjacent outer bumper element, said first and second securing members each further including a cylinder portion extending from said disc portion engaged to said connecting element.

6. The construct of claim 5, wherein said cylinder portion is integrally formed with said disc portion.

7. The construct of claim 1, wherein said first and second intermediate bumpers elements each include an extension portion along said respective mounting element and said end portion extends from said respective mounting element along said longitudinal axis, wherein said end portions each include a spherical shape.

8. The construct of claim 1, wherein said connector assembly includes a first connector portion extending from said first anchor to an inner end of said end portion of said first intermediate bumper element and a second connector portion extending from said second anchor to an inner end of said end portion of said second intermediate bumper element, each of said connector portions including a length between said first and second bone anchors along said longitudinal axis.

9. The construct of claim 8, wherein said length is the same for each of said first and second connector portions.

10. The construct of claim 8, wherein said length is different for each of said first and second connector portions.

11. The construct of claim 1, wherein said end portions of said first and second intermediate bumper elements are spherical and in abutting engagement with one another and said connecting element extends through aligned passages of said abutting end portions.

12. The construct of claim 11, wherein said first and second mounting elements are rigid and substantially incompressible and said first and second intermediate bumper elements and said first and second outer bumper elements are compressible in response to loading from spinal motion of the first and second vertebral bodies.

13. The construct of claim 1, wherein said first and second outer bumper elements each include a cylindrical shape extending around said respective mounting element.

14. The construct of claim 1, further comprising first and second engaging members engageable with said receiver portion of a respective one of said first and second bone anchors to secure said respective mounting element therein.

15. The construct of claim 14, wherein said first and second engaging members rigidly fix said mounting element in said receiver portion of said respective bone anchor.

16. The construct of claim 1, wherein each of said first and second intermediate bumper elements and each of said first and second outer bumper elements are axially compressible in response to loading from spinal motion of the first and second vertebral bodies.

17. The construct of claim 16, wherein said first and second intermediate bumper elements dynamically resist movement of said first and second bone anchors toward one another and said connecting element and said first and second outer bumper elements dynamically resist movement of said first and second bone anchors away from one another.

18. The construct of claim 1, wherein said outer bumper elements and said intermediate bumper elements are positioned in abutting engagement with said respective one of said first and second bone anchors.

19. A spinal stabilization construct, comprising:
first and second bone anchors, said first and second bone anchors each including a proximal receiver portion with a passage and a distal portion engageable to respective ones of first and second vertebral bodies;
a connector assembly extending along a longitudinal axis between said proximal receiver portions of said first and second bone anchors, said connector assembly including:
first and second mounting elements positioned in said passage of a respective one of said first and second bone anchors, each of said mounting elements including a body extending along said longitudinal axis between an inner end located between said first and second bone anchors and an opposite outer end;
first and second intermediate bumper elements extending from a respective one of said first and second mounting elements, each of said first and second intermediate bumper elements including an inner end in abutting engagement with said inner end of the other of said first and second intermediate bumper elements; and
a connecting element extending through said first and second mounting elements and said first and second intermediate bumper elements, said connecting element being elastic and tensioned to maintain a compression force between said first and second bone anchors and said first and second intermediate bumper elements.

20. The construct of claim 19, wherein said connecting element is secured to a respective one of first and second outer bumper elements extending about said outer end of a respective one of said first and second mounting elements.

21. The construct of claim 19, wherein said inner ends of said first and second intermediate bumper elements are pivotal relative to one another in response to movement of said first and second bone anchors relative to one another.

22. The construct of claim 21, wherein said inner ends are formed by spherical end portions of said first and second intermediate bumper elements.

23. The construct of claim 22, wherein said spherical end portions each include a passage extending axially therethrough and said connecting element extends through said passages along said longitudinal axis.

24. The construct of claim 19, wherein said first and second intermediate bumper elements are compressible and said first and second mounting elements are rigid and fixedly engaged to respective ones of said first and second bone anchors.

25. The construct of claim 19, further comprising a first outer bumper element located in engagement with said outer end of said first mounting element and a second outer bumper element located in engagement with said outer end of said second mounting element, wherein said first intermediate bumper element is located in abutting engagement with said first anchor opposite said first outer bumper element and said second intermediate bumper element is located in abutting engagement with said second anchor opposite said second outer bumper element.

26. The construct of claim 25, wherein said first and second intermediate bumper elements each have a stiffness that is greater than a stiffness of each of said first and second outer bumper elements.

27. The construct of claim 19, wherein each of said first and second intermediate bumper elements including a first end in abutting engagement with a respective one of said first and second bone anchors.

28. The construct of claim 19, further comprising a first outer bumper element engaged to said outer end of said first mounting element and located on a side of said first bone anchor opposite said first intermediate bumper element and a second outer bumper element engaged to said outer end of said second mounting element and located on a side of said second bone anchor opposite said second intermediate bumper element.

29. The construct of claims 28, wherein said first outer bumper element extends around said outer end of said first mounting element and said second outer bumper element extends around said outer end of said second mounting element.

30. A method for assembling a spinal stabilization construct, comprising:
providing a first bone anchor spaced from a second bone anchor;
positioning a first connector portion on the first bone anchor and a second connector portion on the second bone anchor, wherein each of the first and second connector portions includes respective ones of first and second intermediate bumper elements extending away from one another from an inner end thereof situated between the first and second bone anchors;
providing a connecting element extending through the first and second connector portions; and
tensioning the connecting element and securing it to the first and second connector portions to compress the first and second bone anchors with the inner ends of the first and second intermediate bumper elements in abutting and pivotal contact with one another.

31. The method of claim 30, wherein each of the connector portions includes a mounting element extending through the respective first and second bone anchor to an outer end of the mounting element, wherein tensioning the connecting element includes securing first and second compressible outer bumper elements against a respective one of the outer ends of the mounting elements.

32. The method of claim 31, wherein the outer bumper elements have a stiffness that differs from a stiffness of the intermediate bumper elements.

33. The method of claim 32, wherein the outer bumper elements have a stiffness that is less than a stiffness of the intermediate bumper elements.

34. The method of claim 30, wherein the first and second connector portions each include a length extending from the inner end of the intermediate bumper element thereof to an opposite outer end of the respective connector portion, and further comprising providing a plurality of connector portions of differing lengths and selecting first and second connector portions forming a desired spacing between the first and second bone anchors when the first and second connector portions are positioned on the first and second bone anchors.

35. The method of claim 34, wherein the intermediate bumper element of each of the plurality of connector portions each includes a spherical end portion adjacent the inner end thereof and the spherical end portions are positioned in abutting and pivotal contact with one another.

36. The method of claim 30, further comprising engaging the first and second bone anchors to respective ones of first and second vertebrae.
